(19) United States
(12) Patent Application Publication
(10) Pub. No.: US 2022/0380716 A1
(43) Pub. Date: Dec. 1, 2022

(54) KITS AND METHODS FOR TEMPERATURE MAINTENANCE AND SHIPMENT INTEGRITY OF CELL CULTURES DURING TRANSPORTATION

(71) Applicant: CORNING INCORPORATED, CORNING, NY (US)

(72) Inventors: Li Cao, Hollis, NH (US); Kirsten Lee Cooper, Waltham, MA (US); Feng Li, Shrewsbury, MA (US); Sweta Nimesh Parikh, Salem, NH (US); Rongjun Zuo, West Roxbury, MA (US)

(21) Appl. No.: 17/642,027

(22) PCT Filed: Sep. 11, 2020
(86) PCT No.: PCT/US2020/050281

§ 371 Date: Mar. 10, 2022

Related U.S. Application Data
(60) Provisional application No. 62/899,545, filed on Sep. 12, 2019.
(60) Provisional application No. 63/043,186, filed on Jun. 24, 2020.

(51) Int. Cl. C12M 1/00; C12M 1/32; C12M 1/12; B65D 81/18; B65D 81/38; B65D 81/02

(52) U.S. Cl. CPC C12M 41/14; C12M 23/12; C12M 23/52; C12M 37/04; B65D 81/18; B65D 81/3813; B65D 81/022

(57) ABSTRACT

A kit for temperature maintenance and shipment integrity of a cell culture is provided, including a well plate, a cell culture, culture media, an insulated receptacle, and a temperature maintenance element. The kit may include a heat release element. The well plate includes a plurality of wells. The cell culture is arranged within each of the plurality of wells of the well plate. Culture media is arranged within each of the plurality of wells of the well plate, where the culture media encapsulates the cell culture. The well plate is arranged within the insulated receptacle. The temperature maintenance element is arranged within the insulated receptacle. The heat release element is also arranged within the insulated receptacle.

### CROSS REFERENCE TO RELATED APPLICATIONS

This application claims the benefit of priority under 35 U.S.C. § 120 of U.S. Provisional Application Ser. No. 63/043,186 filed on Jun. 24, 2020, and U.S. Provisional Application Ser. No. 62/899,545 filed on Sep. 12, 2019, the contents of which are relied upon and incorporated herein by reference in their entireties.

### TECHNICAL FIELD

The present specification generally relates to devices, kits, and methods for temperature maintenance of cell cultures and, more specifically, to devices, kits, and methods for temperature maintenance and shipment integrity of cell cultures during transportation of the cell cultures.

### BACKGROUND

Cell cultures, such as liver spheroids, can be used for drug testing purposes for pharmaceutical applications. However, the cell cultures themselves can be sensitive to temperature, CO2 levels, and other stresses imparted to the cell cultures, all of which affect the viability of the cell culture. Cell cultures can be developed in-house or, if in-house development is not feasible for a company, delivered to the company via a specialty courier that picks up and delivers the cell cultures directly to the company. Specialty couriers can be expensive and cost prohibitive to a company. Additionally, specialty couriers can require specialized transportation equipment, such as a temperature-controlled truck, in order to keep the cell cultures viable during transportation. Traditional shipping methods (i.e., USPS, UPS, FedEx) may be utilized, since these methods could deliver the cell cultures within the required period (i.e., overnight shipping). However, traditional shipping methods could impart too much stress and shock to the cell culture from normal package handling and sorting, greatly compromising the viability of the cell culture. Additionally, these traditional shipping methods do not include temperature regulation, which would also compromise the viability of the cell culture.

Accordingly, a need exists for alternative temperature maintenance and shipment integrity devices, kits, and methods that can keep a cell culture viable during transportation.

### SUMMARY

According to a first aspect, a kit for temperature maintenance of cell cultures may include a well plate, a cell culture, culture media, an insulated receptacle, and a temperature maintenance element. The well plate may include a plurality of wells. The cell culture may be arranged within each of the plurality of wells of the well plate. Culture media may be arranged within each of the plurality of wells of the well plate, where the culture media encapsulates the cell culture. The well plate may be arranged within the insulated receptacle, and the temperature maintenance element may also be arranged within the insulated receptacle.

Another aspect includes a kit according to any of the previous aspects, further comprising a sealing member secured to the well plate, wherein the sealing member may fluidly seal each of the plurality of wells of the well plate.

Another aspect includes a kit according to any of the previous aspects, the sealing member may be adhesively secured to the well plate.

Another aspect includes a kit according to any of the previous aspects, further comprising a gasket arranged on the well plate, where the sealing member may be disposed between the gasket and the well plate.

Another aspect includes a kit according to any of the previous aspects, further comprising a lid arranged on the well plate, where the gasket may be disposed between the sealing member and the lid.

Another aspect includes a kit according to any of the previous aspects, further comprising a sterile container and a shock absorbing container. The well plate may be arranged within the sterile container, and the sterile container and the well plate may be arranged within the shock-absorbing container. The shock-absorbing container may be arranged within the insulated receptacle.

Another aspect includes a kit according to any of the previous aspects, further comprising a collection container, where multiple well plates may be arranged within the collection container, and the collection container may be arranged within the insulated receptacle.

Another aspect includes a kit according to any of the previous aspects, the temperature maintenance element may comprise a plurality of gel packs heated to a pre-shipping temperature prior to being arranged within the insulated receptacle.

Another aspect includes a kit according to any of the previous aspects, the pre-shipping temperature is about 37° C. In some embodiments, the pre-shipping temperature is within a range of about 35° C.-about 40° C. In some embodiments, the pre-shipping temperature is within a range of about 37° C.-about 40° C.

Another aspect includes a kit according to any of the previous aspects, further comprising a heat release element arranged within the insulated receptacle.

Another aspect includes a kit according to any of the previous aspects, the heat release element may comprise a plurality of incubation packs, a plurality of heating packs, or a combination thereof.

Another aspect includes a kit according to any of the previous aspects, the incubation packs may comprise phase change material incubation packs.

Another aspect includes a kit according to any of the previous aspects, the heating packs may comprise oxygen-activated heating packs.

According to a second aspect, a method of maintaining the temperature of a cell culture may include arranging cell cultures within each of a plurality of wells of a well plate. A culture media may be arranged within each of the plurality of wells of the well plate. The culture media encapsulates the cell cultures within the wells. A temperature maintenance element may be arranged within an insulated receptacle, and the well plate may also be within the insulated receptacle.

Another aspect includes a method according to any of the previous aspects, further comprising sealing the well plate with a sealing member, where the sealing member fluidly seals each of the plurality of wells of the well plate. A gasket may be arranged on the well plate, where the sealing member is disposed between the gasket and the well plate. A lid may be secured on the well plate, where the gasket is disposed between the sealing member and the lid.

Another aspect includes a method according to any of the previous aspects, further comprising sealing the well plate within a sterile container and sealing the well plate and sterile container within a shock-absorbing container. The shock-absorbing container may be arranged within the insulated receptacle.

According to a third aspect, a method of maintaining the integrity of a cell culture during transportation may include arranging cell cultures within each of a plurality of wells of a well plate. A culture media may be arranged within each of the plurality of wells of the well plate. The culture media encapsulates the cell cultures within the wells. The well plate may be sealed within a sterile container. The sterile container and well plate may be sealed within a shock-absorbing container. The shock-absorbing container may be arranged within an insulated receptacle. A temperature maintenance element may be arranged within the insulated receptacle, and the well plate may also be within the insulated receptacle.

Another aspect includes a method according to any of the previous aspects, further comprising arranging a heat release element within the insulated receptacle, the heat release element disposed between the temperature maintenance element and the well plate.

Another aspect includes a method according to any of the previous aspects, further comprising arranging a temperature logger within the insulated receptacle and arranging a shock-absorbing material on the temperature maintenance element within the insulated receptacle. The insulated receptacle may be sealed and arranged within a shipping container.

Another aspect includes a method according to any of the previous aspects, further comprising warming the well plate, the sterile container, and the shock-absorbing container prior to arranging the well plate within the insulated receptacle.

Another aspect includes a method according to any of the previous aspects, further comprising arranging multiple well plates within a collection container prior to arranging the well plates within the insulated receptacle.

Another aspect includes a method according to any of the previous aspects, the insulated receptacle may be an insulated box, and the temperature maintenance element may comprise a plurality of gel packs heated to a pre-shipping temperature prior to being arranged within the insulated receptacle.

According to a fourth aspect, a method of maintaining the temperature of a cell culture may include arranging cell cultures within each of a plurality of wells of a well plate. A culture media may be arranged within each of the plurality of wells of the well plate. The culture media encapsulates the cell cultures within the wells. A temperature maintenance element may be arranged within an insulated receptacle, a heat release element may be arranged within the insulated receptacle, and the well plate may also be arranged within the insulated receptacle.

Another aspect includes a method according to any of the previous aspects, further comprising sealing the well plate with a sealing member, wherein the sealing member fluidly seals each of the plurality of wells of the well plate. A gasket may be arranged on the well plate, wherein the sealing member is disposed between the gasket and the well plate. A lid may be secured on the well plate, wherein the gasket is disposed between the sealing member and the lid.

Another aspect includes a method according to any of the previous aspects, further comprising sealing the well plate within a sterile container; and sealing the well plate and sterile container within a shock-absorbing container.

Another aspect includes a method according to any of the previous aspects, further comprising arranging a plurality of shock-absorbing containers comprising sterile containers and well plates within a collection container. A temperature logger may be arranged within the collection container.

Another aspect includes a method according to any of the previous aspects, wherein the heat release element is disposed between the temperature maintenance element and the collection container, and wherein the temperature maintenance element is disposed between the heat release element and the insulated receptacle.

Another aspect includes a method according to any of the previous aspects, further comprising arranging a shock-absorbing material on the temperature maintenance element within the insulated receptacle. The insulated receptacle may be sealed. The insulated receptacle may be arranged within a shipping container.

Another aspect includes a method according to any of the previous aspects, further comprising warming the well plate, the sterile container, and the shock-absorbing container prior to arranging the shock-absorbing containers within the collection container.

Another aspect includes a method according to any of the previous aspects, further comprising warming the culture media prior to arranging the culture media within each of the plurality of wells of the well plate.

Another aspect includes a method according to any of the previous aspects, wherein the heat release element comprises a plurality of incubation packs, a plurality of heating packs, or a combination thereof.

Another aspect includes a method according to any of the previous aspects, wherein the incubation packs are warmed, the heat packs are activated, or both, prior to arranging within the insulated receptacle.

Another aspect includes a method according to any of the previous aspects, wherein the temperature maintenance element comprises a plurality of gel packs heated to a pre-shipping temperature prior to being arranged within the insulated receptacle.

Another aspect includes a method according to any of the previous aspects, further comprising warming the culture media prior to arranging the culture media within each of the plurality of wells of the well plate.

Another aspect includes a method according to any of the previous aspects, further comprising sterilizing the gasket prior to arranging the gasket on the sealing member.

Another aspect includes a method according to any of the previous aspects, the pre-shipping temperature is about 37° C. In some embodiments, the pre-shipping temperature is within a range of about 35° C.-about 40° C. In some embodiments, the pre-shipping temperature is within a range of about 37° C.-about 40° C.

Another aspect includes a method according to any of the previous aspects, the cell cultures are liver spheroids.

Additional features and advantages of the embodiments described herein will be set forth in the detailed description which follows, and in part will be readily apparent to those skilled in the art from that description or recognized by practicing the embodiments described herein, including the detailed description which follows, the claims, as well as the appended drawings.

It is to be understood that both the foregoing general description and the following detailed description describe various embodiments and are intended to provide an overview or framework for understanding the nature and character of the claimed subject matter. The accompanying drawings are included to provide a further understanding of the various embodiments and are incorporated into and constitute a part of this specification. The drawings illustrate the various embodiments described herein, and together with the description serve to explain the principles and operations of the claimed subject matter.

### BRIEF DESCRIPTION OF THE DRAWINGS

FIG. 1 is a side perspective view of a well plate assembly according to one or more embodiments shown or described herein.

FIG. 2 is cross-sectional view of the well plate assembly of FIG. 1, taken generally along line 2-2, according to one or more embodiments shown or described herein.

FIG. 3 is a side perspective view of a packaged well plate assembly according to one or more embodiments shown or described herein.

FIG. 4 is cross-sectional view of the packaged well plate assembly of FIG. 3, taken generally along line 4-4, according to one or more embodiments shown or described herein.

FIG. 5 is a side perspective view of a collection container according to one or more embodiments shown or described herein.

FIG. 6 is a side perspective view of a shipping assembly according to one or more embodiments shown or described herein.

FIG. 7 is a flowchart depicting a method of maintaining the temperature of a cell culture according to one or more embodiments shown or described herein.

FIG. 8 is a side cross-sectional view of a shipping assembly according to one or more embodiments shown or described herein.

FIG. 9 is a side cross-sectional view of a shipping assembly according to one or more embodiments shown or described herein.

FIG. 10 is a side cross-sectional view of a shipping assembly according to one or more embodiments shown or described herein.

FIG. 11 is a flowchart depicting a method according to one or more embodiments shown or described herein.

### DETAILED DESCRIPTION

Reference will now be made in detail to embodiments of cell culture stabilization devices, kits, and methods of operating the same, examples of which are illustrated in the accompanying drawings. Whenever possible, the same reference numerals will be used throughout the drawings to refer to the same or like parts.

Embodiments of the temperature maintenance kit for cell cultures may include a well plate, a cell culture, culture media, an insulated receptacle, and a temperature maintenance element. The kit may further include a heat release element. The well plate may include a plurality of wells. The cell culture may be arranged within each of the plurality of wells of the well plate. Culture media may be arranged within each of the plurality of wells of the well plate, where the culture media encapsulates the cell culture. The cell culture may be a live cell culture. The well plate may be arranged within the insulated receptacle, and the temperature maintenance element may also be arranged within the insulated receptacle. The heat release element may also be arranged within the insulated receptacle. A container of cell culture media, such as a bottle of cell culture media, may also be arranged within the insulated receptacle. Various embodiments of temperature maintenance kits and methods for assembling the same will be described herein with specific reference to the appended drawings.

Embodiments according to the present disclosure include components that allow for the temperature maintenance of cell cultures during the transportation of the cell cultures. Additionally, the embodiments of the present disclosure allow for shock-absorption protection of the cell cultures in order to preserve the viability of the cell culture while being transported.

Kits according to embodiments of the invention may be shipped to customer sites via traditional shipping methods, such as by using shipping services like air transportation or ground shipping. However, live cultures may be subject to stress from temperature changes. In some embodiments, kits according to embodiments of the invention account for shipping at different ambient temperature conditions, which may impact the internal temperature of the package or kit. For example, the ambient temperature may be low during winter months. In such a case, if the ambient temperature is about 4° C., the internal temperature of the package for the kit may decrease to about 27° C. after about 21 hours to about 24 hours.

In some embodiments of the invention, temperature maintenance of cell cultures is provided when shipping the cell cultures during low ambient temperature conditions, such as during winter months. To improve the temperature maintenance capacity of the shipping package design, kits according to embodiments of the invention may include an additional packaging material in the form of a heat release element. The additional packing material allows for the release of heat over an extended length of time in order to maintain the internal temperature and avoid stress on the live cultures, which may be sensitive to stresses such as temperature changes. In preferred embodiments, the internal temperature is maintained at about 37° C. to decrease temperature change stresses to the cultures.

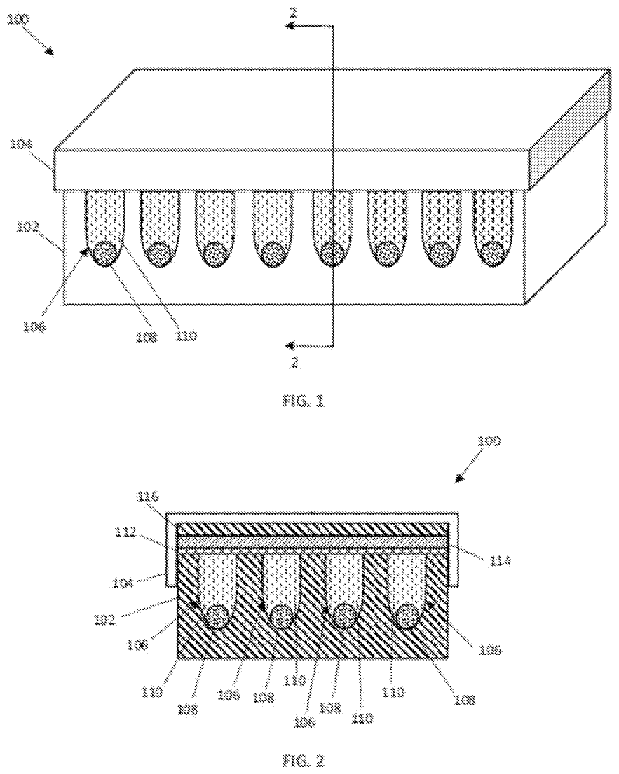

Referring now to FIG. 1, an embodiment of a well plate assembly 100 is schematically depicted. The well plate assembly 100 generally includes a well plate 102 and a lid 104. The well plate 102 includes a plurality of wells 106, with the amount of wells 106 arranged within the well plate 102 varying depending on the size and application of the well plate 102. For example, the well plate 102 may be a ninety-six (96) well, ultra-low attachment surface, sterile spheroid microplate, sold by Corning Incorporated, Catalog no. 4515, 4520. The well plate 102 may be manufactured from glass, plastic, or similar materials. The lid 104 of the well plate assembly 100 may be arranged over the top of the well plate 102 in order to prevent foreign substances from entering the plurality of wells 106. The lid 104 may fit tightly on the well plate 102, or may be secured to the well plate 102 through alternative means, such as tape, elastic bands or the like.

A cell culture 108 may be arranged within each of the plurality of wells 106. In some embodiments, the cell culture 108 is a live cell culture. In some embodiments, the cell culture 108 has a pre-shipping temperature close to about 37° C. In some embodiments, the pre-shipping temperature of the cell culture is in a range of about 35° C. to about 40° C. In some embodiments, the pre-shipping temperature of the cell culture is in a range of about 37° C. to about 40° C. In some embodiments, the cell culture 108 is a liver spheroid which is made from primary human hepatocytes (PHH). However, it should be understood that other types of cell cultures are contemplated and possible. Additionally, a culture media 110 may also be placed within the wells 106 with the cell culture 108.

In the depicted embodiment in FIG. 1, the well plate 102 includes a cell culture 108 arranged within each of the wells 106. The cell culture 108 may have formed a spheroid within the well plate 102 after being developed for a period of time (i.e., ˜6 days). The cell culture 108 may be developed by placing the well plate 102 within an incubator (not shown) set to a temperature within a range of about 35° C.-about 40° C., a range of about 37° C.-about 40° C., or close to about 37° C., with an atmosphere comprising a 5% CO2 concentration. In some embodiments, prior to placing the cell culture 108 within the wells 106, the well plate 102 may be sterilized.

Once the cell culture 108 has formed spheroids, the culture media 110 may be arranged within each of the wells 106 that also contain a cell culture 108. Depending on the type of cell culture 108 being developed within the well plate 102, the culture media 110 may be equilibrated prior to arranging the culture media 110 within the wells 106 containing a cell culture 108. For example, if the cell culture 108 is a liver spheroid, then the culture media 110 may be pre-warmed for two (2) hours within an incubator (not shown) set to a temperature within a range of about 35° C.-about 40° C., a range of about 37° C.-about 40° C., or close to about 37° C., and an atmosphere comprising a 5% CO2 concentration. Once the culture media 110 is equilibrated to the viability conditions of the cell culture 108, a specific volume of the culture media 110 is placed within the wells 106 with the cell culture 108. For example, if the well plate 102 has ninety-six (96) wells, then approximately 300 μL of culture media 110 may be arranged in each well 106. As the culture media 110 is arranged within the wells 106, the culture media 110 encapsulates the cell culture 108. In other words, the culture media 110 prevents foreign objects and contamination from reaching the cell culture 108 and removes any air pockets present in the wells 106.

Referring now to FIG. 2, the well plate assembly 100 may include additional seals in addition to the lid 104. For example, in embodiments, the well plate assembly 100 generally includes an adhesive layer 112, a sealing member 114, and a gasket 116. The sealing member 114 may be a flexible film which is sterile and includes adhesive layer 112 already applied to the sealing member 114. For example, the adhesive layer 112 and the sealing member 114 may be, for example and without limitation, a 6″×3.25″ (6 inch by 3.25 inch) adhesive film sold by Titer-Tops, Catalog no. 490007-086. Additionally, the gasket 116 may be a 0.062″ (0.062 inch) transparent silicon gasket sold by Stockwell Elastomerics, Catalog no. HT6240-57614.

In the embodiment depicted in FIG. 2, after the culture media 110 is arranged within the wells 106 with the cell culture 108, the well plate 102 may be sealed via the sealing member 114. The sealing member 114 is arranged over the well plate 102 and is adhered to the well plate 102 via the adhesive layer 112. The sealing member 114 may be pressed onto the well plate 102, and further rolled to remove any air bubbles that may have formed between the sealing member 114 and the well plate 102. Additionally, a plate presser (not shown) may be used to further seal the edges of the sealing member 114 to the well plate 102. Once adhered, the sealing member 114 and adhesive layer 112 fluidly seal each of the plurality of wells 106 of the well plate 102 from one another.

Referring still to the embodiment depicted in FIG. 2, the gasket 116 may be arranged on top of the well plate 102 such that the sealing member 114 is disposed between the gasket 116 and the well plate 102. The gasket 116 may be sterilized prior to arranging the gasket 116 on the sealing member 114. The gasket 116 may be a soft silicon gasket, which further seals the well plate 102 from foreign contaminants, and absorbs any stresses or shock imparted to the well plate assembly 100. Certain cell cultures 108 may lose viability if stress or shock is imparted to the cell culture 108, and not absorbed by surrounding components. Once the gasket 116 is arranged on the sealing member 114, the lid 104 may be arranged on top of the well plate 102 such that the gasket 116 is disposed between the sealing member 114 and the lid 104. The lid 104 further seals the well plate assembly 100 from foreign contaminants. Additionally, the lid 104 may prevent the sealing member 114 and the gasket 116 from becoming dislodged or accidently removed during transportation of the well plate assembly 100.

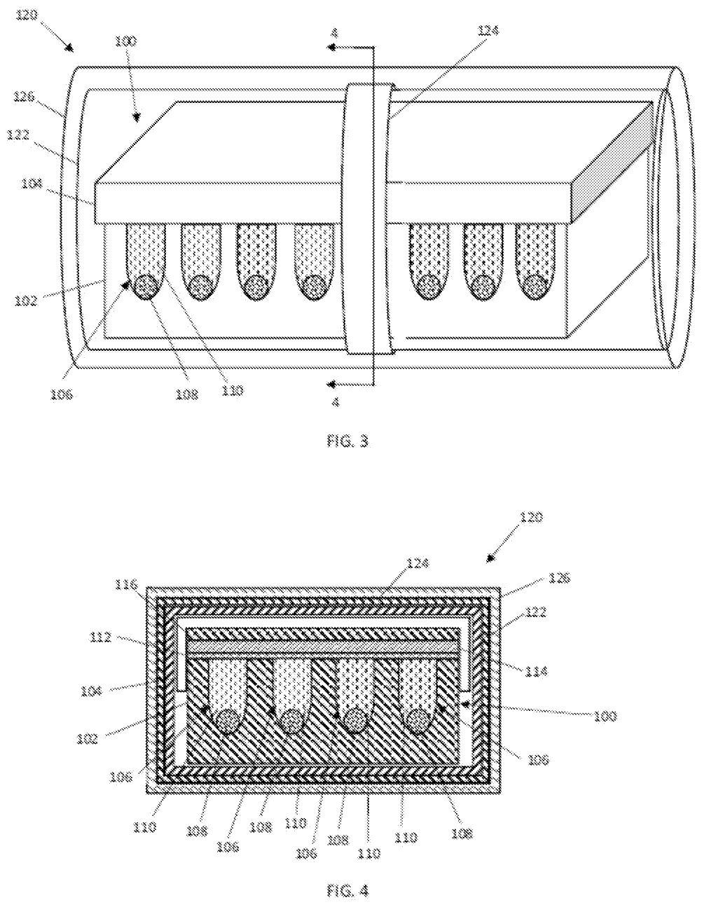

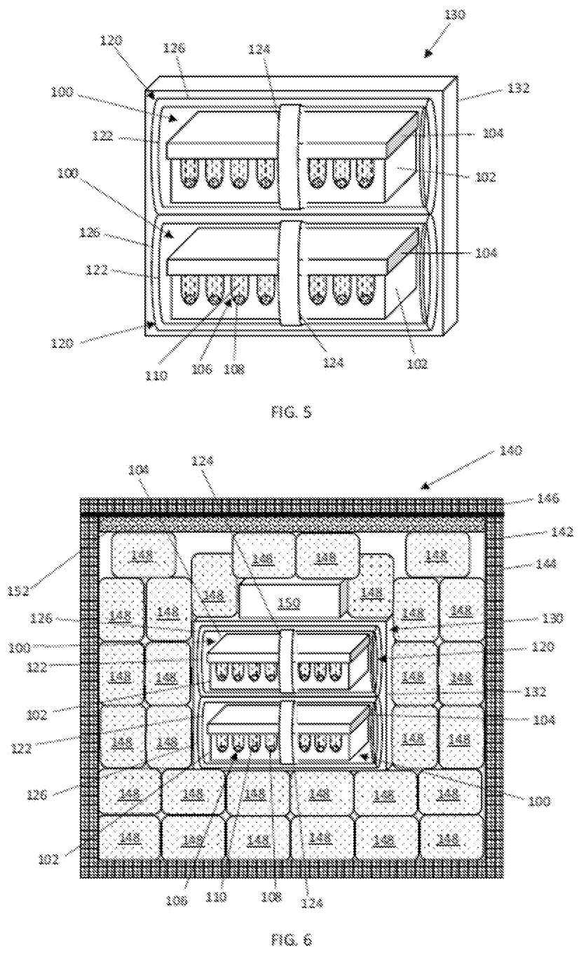

Referring now to FIGS. 3 and 4, an embodiment of a packaged well plate assembly 120 is schematically depicted. The packaged well plate assembly 120 generally includes a well plate assembly 100, a sterile container 122, a sealing strap 124, and a shock-absorbing container 126.

In the embodiment depicted in FIGS. 3 and 4, the well plate assembly 100, which has been sealed by the lid 104, sealing member 114, and gasket 116, is arranged within the sterile container 122. The sterile container 122 may further prevent foreign contamination of the cell culture 108, while also preventing excessive leakage if the lid 104, sealing member 114, and/or gasket 116 fail to fluidly seal the plurality of wells 106 of the well plate 102. The sterile container 122 may be, for example and without limitation, a sterile plastic bag, such as a 6″×9″ (6 inch by 9 inch) sterile Whirl-Pak® plastic bag sold by Nasco, Catalog no. 11216-409. Once the well plate assembly 100 is arranged within the sterile container 122, the sterile container 122 is sealed using the sealing strap 124. The sealing strap 124 may be separate or integral with the sterile container 122 and may include an adhesive surface (not shown) which allows the sealing strap 124 to adhere to itself or a portion of the sterile container 122 in order to seal the sterile container 122.

Referring still to the embodiment depicted in FIGS. 3 and 4, once the well plate assembly 100 is sealed within the sterile container 122, the well plate assembly 100 and the sterile container 122 may be arranged within the shock-absorbing container 126. The shock-absorbing container 126 absorbs stresses or shock imparted to the well plate assembly 100 during transportation of the well plate assembly 100. As stated previously, the cell culture 108 may be susceptible to damage from stresses and shock imparted to the well plate 102, which would decrease the viability of the cell culture 108. The shock-absorbing container 126 may be any form of shock-absorbing material or design, for example, a flexible bag lined with bubble wrap. Once the well plate assembly 100 and the sterile container 122 are arranged within the shock-absorbing container 126, the shock-absorbing container 126 is sealed, forming a packaged well plate assembly 120. The packaged well plate assembly 120 may be placed within an incubator (not shown) set to a temperature within a range of about 37° C.-about 40° C., a range of about 37° C.-about 40° C., or close to about 37° C., and an atmosphere comprising a 5% CO2 concentration in order to prepare the cell culture 108 for further packaging, since the cell culture 108 has been removed from the incubator for a period of time and has cooled own during the assembly of the packaged well plate assembly 120.

In some embodiments, an absorbent material (not shown) may be placed within the shock-absorbing container 126. The absorbent material may be used to absorb spills or leakage of the cell culture 108 and/or the culture media 110 if the lid 104, sealing member 114, gasket 116, and/or sterile container 122 failed to properly seal the plurality of wells 106 of the well plate 102.

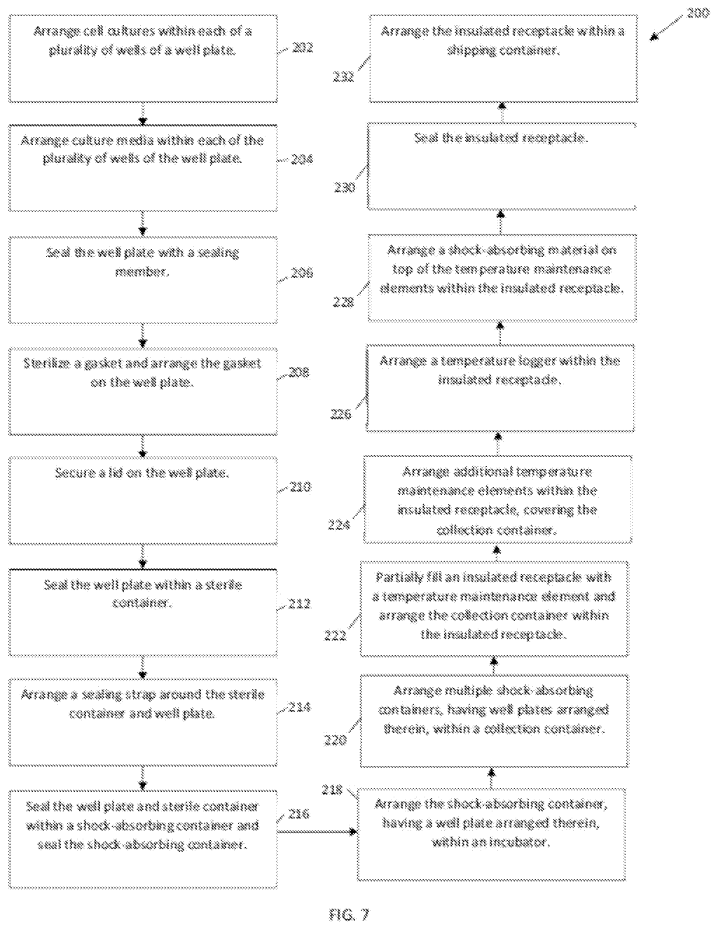

Referring now to FIG. 5, an embodiment of a collection container assembly 130 is schematically depicted. The collection container assembly 130 generally includes at least one packaged well plate assembly 120 arranged within a collection container 132. The collection container 132 may contain multiple packaged well plate assemblies 120. The collection container 132 may be any container which can fit multiple packaged well plate assemblies 120 and prevent the packaged well plate assemblies 120 from shifting or moving during transportation of the collection container assembly 130. In embodiments, the collection container 132 may be, for example and without limitation, a cardboard box. The collection container 132 may be sized to fit a specific amount of packaged well plate assemblies 120 with little to no extra space within the collection container 132 in order to prevent shifting of the packaged well plate assemblies 120. Additionally, the collection container 132 may include shock-absorbing properties to further protect the cell culture 108 from shock and impact stresses during transportation of the cell culture 108.

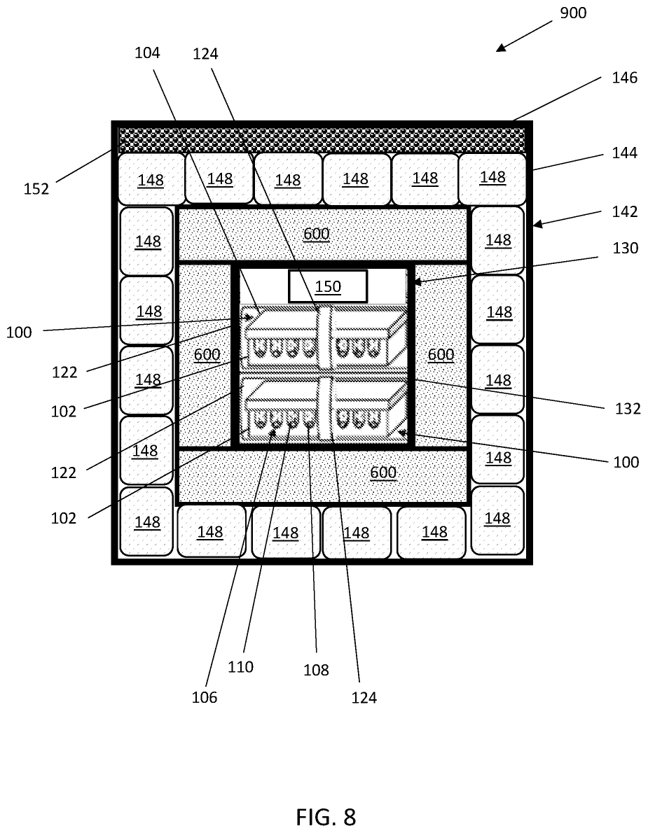

Referring now to FIG. 6, an embodiment of an insulated receptacle assembly 140 is schematically depicted. The insulated receptacle assembly 140 generally includes a collection container assembly 130, an insulated receptacle 142, at least one temperature maintenance element 148, a temperature logger 150, and a shock-absorbing material 152. The insulated receptacle 142 includes a box 144 and a lid 146. The collection container assembly 130, at least one temperature maintenance element 148, and temperature logger 150 may all be arranged within the insulated receptacle 142.

In embodiments, the insulated receptacle 142 may be formed of an insulating material, or be lined with an insulating material, such as Styrofoam. The lid 146 may fit tightly on the box 144, or may be secured to the box 144 via another securement means, such as tape. The insulated receptacle 142 may insulate the cell culture 108 during transportation in order to maintain the cell culture 108 at or above a specific viability temperature related to the cell culture 108. For example, in the case of liver spheroid cell cultures, the liver spheroids may remain viable at 27° C. for up to eight (8) hours, or the liver spheroids may remain viable at 22° C. for up to four (4) hours.

Referring still to the embodiment depicted in FIG. 6, the temperature maintenance element 148 may be a plurality of temperature maintenance elements 148. The temperature maintenance elements 148 may be gel packets which can be pre-warmed in an incubator (not shown) to a pre-shipping temperature in order to prepare for packaging into the insulated receptacle 142. In embodiments, the pre-shipping temperature may be a temperature within a range of about 35° C.-about 40° C., a range of about 37° C.-about 40° C., or close to about 37° C., and is related to a viability temperature of the cell culture 108 and the transportation period. The temperature maintenance elements 148 may also provide cushioning and shock-absorbing benefits, such as when flexible temperature maintenance elements are utilized, such as thermal gel packs which can deform under pressure. In embodiments, the temperature maintenance elements 148 are Thermosafe 406J gel packets available from Cole Parmer, Catalog no. U-06-345-60. However, it should be understood that other temperature maintenance elements are contemplated and possible.

The temperature logger 150 may be any form of temperature reading device. The temperature logger 150 may be placed within the insulated receptacle 142 prior to sealing the insulated receptacle 142. The temperature logger 150 measures the internal temperature of the insulated receptacle 142 during transportation of the cell culture 108. Upon arrival to a destination, the temperature logger 150 can be observed to ensure that the internal temperature of the insulated receptacle 142 did not drop below a viability temperature of the cell culture 108 during transportation. In embodiments, the temperature logger 150 is a TT Ultra Multi Use temperature logger from Sensitech, Catalog no. TUA02-01-033. However, it should be understood that other temperature loggers are contemplated and possible.

The shock-absorbing material 152 may be any form of shock-absorbing material, such as bubble wrap or foam. After the collection container assembly 130, the at least one temperature maintenance element 148, and the temperature logger 150 are arranged within the insulated receptacle 142, the shock-absorbing material 152 is arranged within the box 144 and over the components arranged within the insulated receptacle 142. The shock-absorbing material 152 may also provide additional insulation properties to the insulated receptacle assembly 140.

Referring still to the embodiment depicted in FIG. 6, the insulated receptacle assembly 140 is assembled to provide temperature maintenance and shock-absorption to the cell culture 108. The plurality of temperature maintenance elements 148 are arranged in the bottom of the insulated receptacle 142. The plurality of temperature maintenance elements 148 have been pre-warmed to a pre-shipping temperature, which is equal to or higher than the viability temperature of the cell culture 108. The collection container assembly 130, which includes the cell culture 108, is also placed within the insulated receptacle 142, on top of the plurality of temperature maintenance elements 148. Additionally, temperature maintenance elements 148 are also arranged around the collection container assembly 130, filling the insulated receptacle 142 to slightly below the maximum holding volume of the insulated receptacle 142. The temperature logger 150 is also arranged within the insulated receptacle 142, on top of the plurality of temperature maintenance elements 148. A container or bottle of cell culture media (not shown) may also be arranged within the insulated receptacle 142, and in some embodiments, may be disposed on top of the plurality of temperature maintenance elements 148.

Any additional temperature maintenance elements 148 are placed into the insulated receptacle 142, with the shock-absorbing material 152 being arranged within the insulated receptacle 142 in order to fill the insulated receptacle 142 to its maximum holding volume. The lid 146 is placed on the box 144, sealing the insulated receptacle 142, and forming the insulated receptacle assembly 140. By filling the insulated receptacle 142 to its maximum volume using the plurality of temperature maintenance elements 148, the cell culture 108 will be further insulated and protected against shock imparted to the insulated receptacle assembly 140 since there is little to no room for the collection container assembly 130 to shift or adjust within the insulated receptacle 142.

Referring still to the embodiment depicted in FIG. 6, the insulated receptacle assembly 140 may be subject to a package and plate packing integrity test performed in accordance with the Package Design Verification Protocol for ISTA 1A. The ISTA 1A protocol includes placing two (2) packaged well plate assemblies 120 within a collection container 132 to form a collection container assembly 130, then placing three (3) collection container assemblies 130, along with a 100 mL bottle of culture media 110 and the temperature logger 150, within the insulated receptacle 142. The insulated receptacle 142 is then filled with a plurality of temperature maintenance elements 148, sealed shut, and placed inside a shipping container. The shipping container is then sealed with tape. The shipping container and insulated receptacle assembly 140 are then subject to various tests, including a vibration test and a 10-point shock test. The well plate assembly 100, packaged well plate assembly 120, collection container assembly 130, and insulated receptacle assembly 140 may all pass the ISTA 1A protocol with little to no visible damage or leakage.

Referring now to FIG. 7, a flow chart depicting a method 200 for maintaining the temperature of a cell culture 108 (as illustrated in FIGS. 1-6) is depicted. It is noted that while a number of steps are shown in a specific order, embodiments may include a greater or fewer number of steps in varying orders without departing from the scope of the present disclosure. To begin, at step 202 the method 200 may include arranging the cell culture 108 within each of the plurality of wells 106 of the well plate 102. For example, and with reference to FIGS. 1-6, the cell culture 108 are developed within the well plate 102 for a period prior to beginning to package the cell culture 108 for shipping.

Referring again to FIG. 7, step 204 may include arranging culture media 110 within each of the plurality of wells 106 of the well plate 102. For example, and as described above, the culture media 110 may be pre-warmed in an incubator (not shown) to a viability temperature of the cell culture 108 prior to being arranged in the wells 106. Step 206 may include sealing the well plate 102 with a sealing member 114, where the sealing member 114 fluidly seals each of the plurality of wells 106 of the well plate 102. For example, and with reference to FIG. 2, the sealing member 114 is adhered to the well plate 102 via the adhesive layer 112.

Referring again to FIG. 7, step 208 may include arranging the gasket 116 on the well plate 102, where the sealing member 114 is disposed between the gasket 116 and the well plate 102. For example, and as described above, the gasket 116 may be arranged on the sealing member 114. The gasket 116 may be separately sterilized prior to being arranged on the well plate 102. Step 210 may include securing the lid 104 on the well plate 102, where the gasket 116 is disposed between the sealing member 114 and the lid 104.

Referring again to FIG. 7, step 212 may include sealing the well plate 102 within a sterile container 122. For example, and with reference to FIGS. 3 and 4, the sterile container 122 may be a plastic bag which encompasses the well plate 102 to stop the spread of any leaks from the wells 106 during transportation. Step 214 may include arranging a sealing strap 124 around the well plate 102 and the sterile container 122. For example, the sealing strap 124 may be a self-adhesive strap that adheres to itself or the sterile container 122 in order to seal the sterile container 122.

Referring again to FIG. 7, step 216 may include sealing the well plate 102 and sterile container 122 within a shock-absorbing container 126. For example, the shock-absorbing container 126 may be any form of shock-absorbing material, such as foam or bubble wrap. Step 218 may include warming the well plate 102, the sterile container 122, and the shock-absorbing container 126 prior to arranging the well plate 102 within the insulated receptacle 142. For example, and as described above, during the sealing process, the cell culture 108 may become cooled down since the well plate 102 is in an ambient temperature environment. In order to protect viability of the cell culture 108, the packaged well plate assembly 120 may be placed into an incubator (not shown) in order to bring all the components back up to the viability temperature of the cell culture 108.

Referring again to FIG. 7, step 220 may include arranging multiple well plates 102 within the collection container 132 prior to arranging the well plates 102 within the insulated receptacle 142. For example, the collection container 132 may be utilized to further protect the cell culture 108 from shifting during transportation of the cell culture 108. Step 222 may include arranging at least one temperature maintenance element 148 within the insulated receptacle 142 and arranging the collection container 132 within the insulated receptacle 142. For example, and as described above, the temperature maintenance element 148 may be pre-warmed to a pre-shipping temperature, which is equal to or higher than the viability temperature of the cell culture 108.

Referring again to FIG. 7, step 224 may include arranging an additional temperature maintenance element 148 within the insulated receptacle 142, covering the collection container 132. For example, additional temperature maintenance elements 148 may be arranged within the insulated receptacle 142 in order to fill the insulated receptacle 142 to slightly less than its maximum holding volume. Step 226 may include arranging a temperature logger 150 within the insulated receptacle 142. For example, the temperature logger 150 may be arranged on top of the temperature maintenance elements 148, and may measure the internal temperature of the insulated receptacle 142 during transportation of the cell culture 108.

Referring again to FIG. 7, step 228 may include arranging a shock-absorbing material 152 on the temperature maintenance element 148 within the insulated receptacle 142. For example, and as described above, the shock-absorbing material 152 is arranged within the insulated receptacle 142 in order to fill the insulated receptacle 142 to its maximum holding volume. Step 230 may include sealing the insulated receptacle 142 with the lid 146.

Referring again to FIG. 7, step 232 may include arranging the insulated receptacle 142 within a shipping container (not shown). For example, once the insulated receptacle 142 is sealed, the insulated receptacle 142 may be placed in a traditional shipping box, for example a cardboard box, and transported to its destination via traditional shipping methods, such as overnight delivery, without the need for a special courier service.

Referring now to FIG. 8 to FIG. 10, embodiments of insulated receptacle assemblies 900, 920, and 940 are schematically depicted. The insulated receptacle assemblies 900, 920, 940 generally include a collection container assembly 130, an insulated receptacle 142, at least one temperature maintenance element 148, at least one heat release element 600, 700, a temperature logger 150, and a shock-absorbing material 152. The insulated receptacle 142 includes a box 144 and a lid 146. The collection container assembly 130, at least one temperature maintenance element 148, and at least one heat release element 600, 700 may all be arranged within the insulated receptacle 142.

In embodiments, the insulated receptacle 142 may be formed of an insulating material, or be lined with an insulating material, such as Styrofoam. As used herein, “insulating material” refers to a material that is thermally insulating. The lid 146 may fit tightly on the box 144, or may be secured to the box 144 via another securement means, such as tape. The insulated receptacle 142 may insulate the cell culture 108 during transportation in order to maintain the cell culture 108 at or above a specific viability temperature related to the cell culture 108. For example, in the case of liver spheroid cell cultures, the liver spheroids may remain viable at 27° C. for up to eight (8) hours, or the liver spheroids may remain viable at 22° C. for up to four (4) hours.

The temperature maintenance element 148 may be a plurality of temperature maintenance elements 148. The temperature maintenance elements 148 may be gel packets which can be pre-warmed in an incubator (not shown) to a pre-shipping temperature in order to prepare for packaging into the insulated receptacle 142. In embodiments, the pre-shipping temperature may be a temperature within a range of about 35° C.-about 40° C., a range of about 37° C.-about 40° C., or close to about 37° C., and is related to a viability temperature of the cell culture 108 and the transportation period. The temperature maintenance elements 148 may also provide cushioning and shock-absorbing benefits, such as when flexible temperature maintenance elements are utilized, such as thermal gel packs which can deform under pressure. In embodiments, the temperature maintenance elements 148 are Thermosafe 406J gel packets available from Cole Parmer, Catalog no. U-06-345-60. However, it should be understood that other temperature maintenance elements are contemplated and possible.

The temperature logger 150 may be any form of temperature reading device. The temperature logger 150 may be placed within the collection container 132 before the collection container 132 is closed. The temperature logger 150 measures the internal temperature of the collection container 132 during transportation of the cell culture 108. The temperature logger 150 can also store a record of the internal temperature over time, or at least one of a high and low temperature, or can transmit the same for recording elsewhere. Upon arrival to a destination, the temperature logger 150 can be observed to ensure that the internal temperature of the collection container 132 did not drop below a viability temperature of the cell culture 108 during transportation. In embodiments, the temperature logger 150 is a TT Ultra Multi Use temperature logger from Sensitech, Catalog no. TUA02-01-033. However, it should be understood that other temperature loggers are contemplated and possible.

The shock-absorbing material 152 may be any form of shock-absorbing material, such as bubble wrap or foam. After the collection container assembly 130, the at least one temperature maintenance element 148, the at least one heat release element 600, and the temperature logger 150 are arranged within the insulated receptacle 142, the shock-absorbing material 152 is arranged within the box 144 and over the components arranged within the insulated receptacle 142. The shock-absorbing material 152 may also provide additional insulation properties to the insulated receptacle assembly 140.

The insulated receptacle assembly 900, 920, 940 is assembled to provide temperature maintenance and shock-absorption to the cell culture 108. The plurality of temperature maintenance elements 148 are arranged in the bottom of the insulated receptacle 142. The plurality of temperature maintenance elements 148 have been pre-warmed to a pre-shipping temperature, which is equal to or higher than the viability temperature of the cell culture 108.

The heat release element 600, 700 may be any suitable heat release device. For example, the heat release element may comprise an incubation pack 600, a heat pack 700, or a combination thereof. The heat release element 600, 700 may be a plurality of heat release elements 600, 700.

Referring to FIG. 8, the heat release element 600, 700 comprises a plurality of incubation packs 600. The collection container 132 is surrounded by incubation packs 600 on all sides. In some embodiments, the incubation pack 600 is a phase changing material incubation pack. In some embodiments, the incubation packs 600 are warmed in an incubator for about 24 hours prior to packing the shipping container. About two hours prior to packing the shipping container, the incubator temperature is decreased and the incubation packs 600 that are used are packs in a liquid form. In embodiments, the incubation pack is a Phase Change Material (PCM) for thermal energy storage, 37° C. incubation pack from SavEnrg, Catalog no. PCM-OM37P. However, it should be understood that other incubation packs are contemplated and possible.

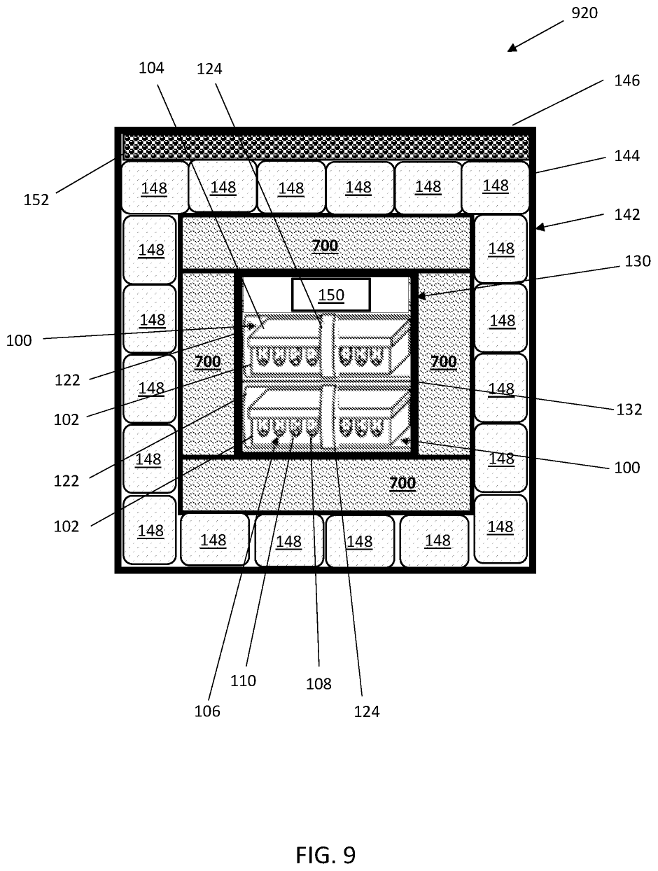

Referring to FIG. 9, the heat release element 600, 700 comprises a plurality of heat packs 700. The collection container 132 is surrounded by heat packs 700 on all sides. In some embodiments, the heat pack 700 is an oxygen-activated heating pack. In some embodiments, the heat packs 700 are opened prior to packing the shipping container in order to allow for air contact and activation. In embodiments, the heat pack is a Techniche Body Warmer, 24 Hours Heating Time, Activates by Contact with Air heat pack from Grainger, Catalog no. 45A274. However, it should be understood that other heat packs are contemplated and possible.

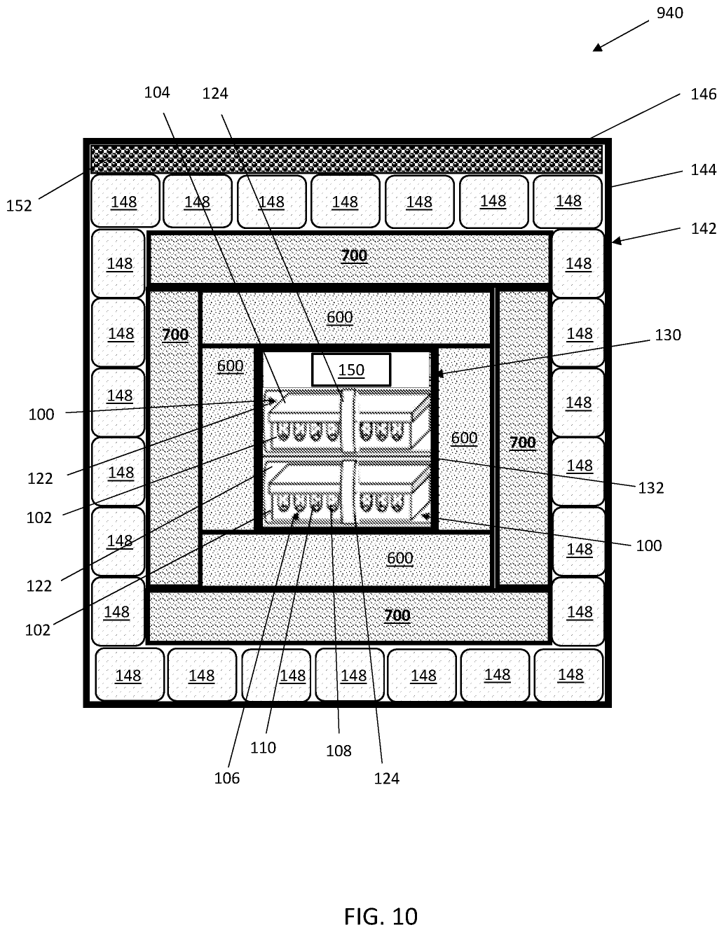

Referring to FIG. 10, the heat release element 600, 700 comprises a plurality of incubation packs 600 and a plurality of heat packs 700. The collection container 132 is surrounded by incubation packs 600 on all sides, and heat packs 700 are disposed around the incubation packs 600.

The plurality of heat release elements 600, 700 are arranged on top of the temperature maintenance elements 148. The collection container assembly 130, which includes the cell culture 108 and temperature logger 150, is also placed within the insulated receptacle 142, on top of the heat release elements 600, 700, which are disposed on top of the plurality of temperature maintenance elements 148. Additionally, heat release elements 600, 700 are also arranged around the collection container assembly 130, and temperature maintenance elements 148 are also arranged around the heat release elements 600, 700, thereby filling the insulated receptacle 142 to slightly below the maximum holding volume of the insulated receptacle 142. A container or bottle of cell culture media (not shown) may also be arranged within the insulated receptacle 142, disposed between the collection container assembly 130 and the shock-absorbing material 152 and between a plurality of temperature maintenance elements 148.

Any additional temperature maintenance elements 148 are placed into the insulated receptacle 142, with the shock-absorbing material 152 being disposed on top of the temperature maintenance elements within the insulated receptacle 142 in order to fill the insulated receptacle 142 to its maximum holding volume. The lid 146 is placed on the box 144, sealing the insulated receptacle 142, and forming the insulated receptacle assembly 900, 920, 940. By filling the insulated receptacle 142 to its maximum volume using the plurality of temperature maintenance elements 148, the cell culture 108 will be further insulated and protected against shock imparted to the insulated receptacle assembly 900, 920, 940 since there is little to no room for the collection container assembly 130 to shift or adjust within the insulated receptacle 142.

The insulated receptacle assembly 900, 920, 940 may be subject to a package and plate packing integrity test performed in accordance with the Package Design Verification Protocol for ISTA 1A. The ISTA 1A protocol includes placing two (2) packaged well plate assemblies 120 within a collection container 132 to form a collection container assembly 130, then placing three (3) collection container assemblies 130, along with a 100 mL bottle of culture media 110 and the temperature logger 150, within the insulated receptacle 142. The insulated receptacle 142 is then filled with a plurality of heat release elements 600, 700, a plurality of temperature maintenance elements 148, sealed shut, and placed inside a shipping container. The shipping container is then sealed with tape. The shipping container and insulated receptacle assembly 900, 920, 940 are then subject to various test including a vibration test and a 10-point shock test. The well plate assembly 100, packaged well plate assembly 120, collection container assembly 130, and insulated receptacle assembly 900, 920, 940 may all pass the ISTA 1A protocol with little to no visible damage or leakage.

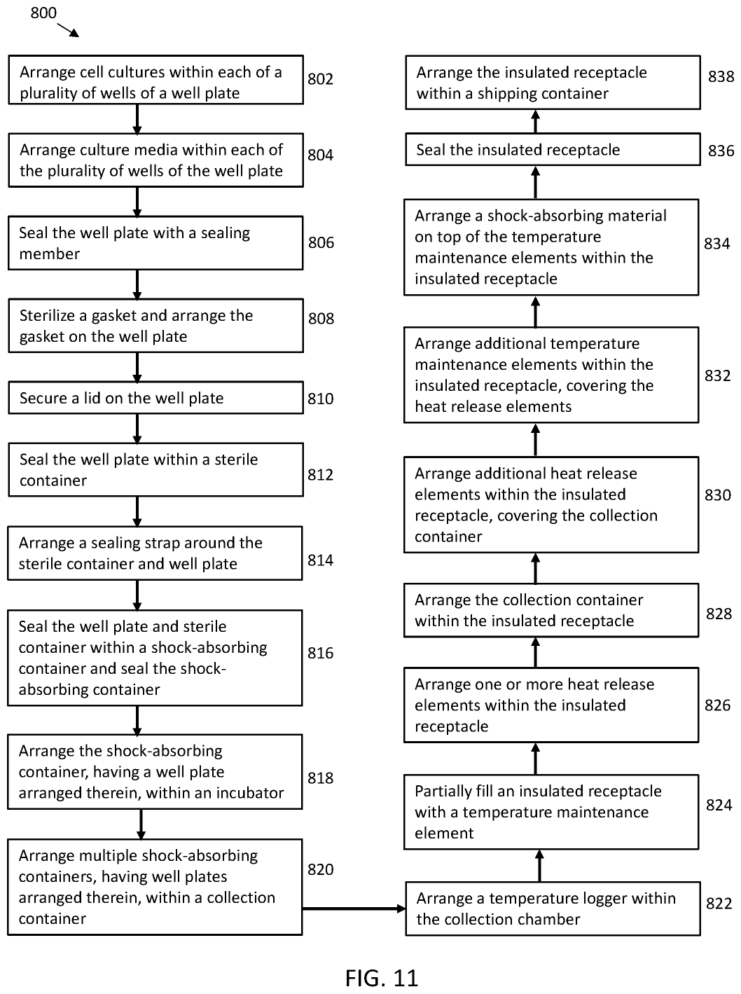

Referring now to FIG. 11, a flow chart depicting a method 800 for maintaining the temperature of a cell culture 108 is depicted. It is noted that while a number of steps are shown in a specific order, embodiments may include a greater or fewer number of steps in varying orders without departing from the scope of the present disclosure. To begin, at step 802 the method 800 may include arranging the cell culture 108 within each of the plurality of wells 106 of the well plate 102. For example, the cell culture 108 is developed within the well plate 102 for a period prior to beginning to package the cell culture 108 for shipping.

Step 804 may include arranging culture media 110 within each of the plurality of wells 106 of the well plate 102. For example, and as described above, the culture media 110 may be pre-warmed in an incubator (not shown) to a viability temperature of the cell culture 108 prior to being arranged in the wells 106. Step 806 may include sealing the well plate 102 with a sealing member 114, where the sealing member 114 fluidly seals each of the plurality of wells 106 of the well plate 102. For example, and with reference to FIG. 2, the sealing member 114 is adhered to the well plate 102 via the adhesive layer 112.

Step 808 may include sterilizing the gasket 116 and arranging the gasket 116 on the well plate 102, where the sealing member 114 is disposed between the gasket 116 and the well plate 102. For example, and as described above, the gasket 116 may be arranged on the sealing member 114. The gasket 116 may be separately sterilized prior to being arranged on the well plate 102. Step 810 may include securing the lid 104 on the well plate 102, where the gasket 116 is disposed between the sealing member 114 and the lid 104.

Step 812 may include sealing the well plate 102 within a sterile container 122. For example, and with reference to FIGS. 3 and 4, the sterile container 122 may be a plastic bag which encompasses the well plate 102 to stop the spread of any leaks from the wells 106 during transportation. Step 814 may include arranging a sealing strap 124 around the well plate 102 and the sterile container 122. For example, the sealing strap 124 may be a self-adhesive strap that adheres to itself or the sterile container 122 in order to seal the sterile container 122.

Step 816 may include sealing the well plate 102 and sterile container 122 within a shock-absorbing container 126. For example, the shock-absorbing container 126 may be any form of shock-absorbing material, such as foam or bubble wrap. Step 818 may include warming the well plate 102, the sterile container 122, and the shock-absorbing container 126 prior to arranging the well plate 102 within the insulated receptacle 142. For example, and as described above, during the sealing process, the cell culture 108 may become cooled down since the well plate 102 is in an ambient temperature environment. In order to protect viability of the cell culture 108, the packaged well plate assembly 120 may be placed into an incubator (not shown) in order to bring all of the components back up to the viability temperature of the cell culture 108.

Step 820 may include arranging multiple well plates 102 in respective shock-absorbing containers 126 within the collection container 132 prior to arranging the well plates 102 within the insulated receptacle 142. For example, the collection container 132 may be utilized to further protect the cell culture 108 from shifting during transportation of the cell culture 108. Step 822 may include arranging a temperature logger 150 within the collection chamber 132. For example, the temperature logger 150 may be arranged within the collection chamber 132 before closing the collection chamber 132. The temperature logger 150 may measure the internal temperature of the collection chamber 132 within the insulated receptacle 142 during transportation of the cell culture 108.

Step 824 may include arranging at least one temperature maintenance element 148 within the insulated receptacle 142. For example, and as described above, the temperature maintenance element 148 may be pre-warmed to a pre-shipping temperature, which is equal to or higher than the viability temperature of the cell culture 108.

Step 826 may include arranging at least one heat release element 600, 700 within the insulated receptacle 142. The heat release element may be an incubation pack 600, heat pack 700, or combination thereof. The incubation pack 600 may be warmed in advance of shipping, and the heat pack 700 may be activated in advance of shipping. Step 828 may include arranging the collection container 132 within the insulated receptacle 142. Step 830 may include arranging additional heat release element 600, 700 within the insulated receptacle 142, covering the collection container 132.

Step 832 may include arranging an additional temperature maintenance element 148 within the insulated receptacle 142, covering the heat release elements 600, 700. For example, additional temperature maintenance elements 148 may be arranged within the insulated receptacle 142 in order to fill the insulated receptacle 142 to slightly less than its maximum holding volume. For example, the temperature maintenance elements 148 are disposed between the insulated receptacle 142 and the heat release elements 600, 700, and the heat release elements 600, 700 are disposed between the temperature maintenance elements 148 and the collection container 132.

Step 834 may include arranging a shock-absorbing material 152 on the temperature maintenance element 148 within the insulated receptacle 142. For example, and as described above, the shock-absorbing material 152 is arranged within the insulated receptacle 142 in order to fill the insulated receptacle 142 to its maximum holding volume. Step 836 may include sealing the insulated receptacle 142 with the lid 146.

Step 838 may include arranging the insulated receptacle 142 within a shipping container (not shown). For example, once the insulated receptacle 142 is sealed, the insulated receptacle 142 may be placed in a traditional shipping box, for example a cardboard box, and transported to its destination via traditional shipping methods, such as overnight delivery or two-day delivery, without the need for a special courier service.

It should now be understood that embodiments described herein are directed to temperature maintenance systems and methods that allow for the temperature maintenance and shock-absorption protection of cell cultures during the transportation of the cell cultures. The temperature maintenance systems include a well plate, a cell culture, culture media, an insulated receptacle, and a temperature maintenance element. The temperature maintenance systems may include a heat release element. The well plate includes a plurality of wells. The cell culture is arranged and developed within each of the plurality of wells of the well plate. Culture media is also arranged within each of the plurality of wells of the well plate, where the culture media encapsulates the cell culture and is utilized to remove any air pockets within the wells. The well plate is arranged within the insulated receptacle. The temperature maintenance element is also arranged within the insulated receptacle. The heat release element is also arranged within the insulated receptacle. The arrangement within the insulated receptacle allows the internal temperature of the insulated receptacle to remain at or above the viability temperature of the cell culture.

It will be apparent to those skilled in the art that various modifications and variations can be made to the embodiments described herein without departing from the spirit and scope of the claimed subject matter. Thus, it is intended that the specification cover the modifications and variations of the various embodiments described herein provided such modification and variations come within the scope of the appended claims and their equivalents.

1. A kit, comprising:
a well plate comprising a plurality of wells;
a cell culture arranged within each of the plurality of wells of the well plate;
culture media arranged within each of the plurality of wells of the well plate, wherein the culture media encapsulates the cell culture;
an insulated receptacle, wherein the well plate is arranged within the insulated receptacle; and
a temperature maintenance element arranged within the insulated receptacle.

2. The kit of claim 1, further comprising a sealing member secured to the well plate, wherein the sealing member fluidly seals each of the plurality of wells of the well plate.

3. (canceled)

4. The kit of claim 2, further comprising a gasket arranged on the well plate, wherein the sealing member is disposed between the gasket and the well plate.

5. The kit of claim 4, further comprising a lid arranged on the well plate, wherein the gasket is disposed between the sealing member and the lid.

6. The kit of claim 5, further comprising:
a sterile container, wherein the well plate is arranged within the sterile container; and
a shock-absorbing container, wherein the sterile container and the well plate are arranged within the shock-absorbing container, and the shock-absorbing container is arranged within the insulated receptacle.

7. The kit of claim 6, further comprising a collection container, wherein multiple well plates are arranged within the collection container, and the collection container is arranged within the insulated receptacle.

8. The kit of claim 1, wherein the temperature maintenance element comprises a plurality of gel packs heated to a pre-shipping temperature prior to being arranged within the insulated receptacle.

9. (canceled)

10. (canceled)

11. The kit of claim 1, further comprising a heat release element arranged within the insulated receptacle, wherein the heat release element comprises a plurality of incubation packs, a plurality of heating packs, or a combination thereof.

12. The kit of claim 11, wherein the incubation packs comprise phase change material incubation packs.

13. The kit of claim 11, wherein the heating packs comprise oxygen-activated heating packs.

14. A method of maintaining the temperature of a cell culture, the method comprising:
arranging cell cultures within each of a plurality of wells of a well plate;
arranging culture media within each of the plurality of wells of the well plate, wherein the culture media encapsulates the cell cultures within the wells;
arranging a temperature maintenance element within an insulated receptacle; and
arranging the well plate within the insulated receptacle.

15. The method of claim 14, further comprising:
sealing the well plate with a sealing member, wherein the sealing member fluidly seals each of the plurality of wells of the well plate;
arranging a gasket on the well plate, wherein the sealing member is disposed between the gasket and the well plate; and
securing a lid on the well plate, wherein the gasket is disposed between the sealing member and the lid.

16. The method of claim 15, further comprising:
sealing the well plate within a sterile container; and
sealing the well plate and sterile container within a shock-absorbing container, wherein the shock-absorbing container is arranged within the insulated receptacle.

17. The method of claim 16, further comprising:
arranging a temperature logger within the insulated receptacle;
arranging a shock-absorbing material on the temperature maintenance element within the insulated receptacle;
sealing the insulated receptacle; and
arranging the insulated receptacle within a shipping container.

18. The method of claim 14, further comprising warming the culture media prior to arranging the culture media within each of the plurality of wells of the well plate.

19. The method of claim 15, further comprising sterilizing the gasket prior to arranging the gasket on the sealing member.

20. The method of claim 16, further comprising warming the well plate, the sterile container, and the shock-absorbing container prior to arranging the well plate within the insulated receptacle.

21. The method of claim 16, further comprising arranging multiple well plates within a collection container prior to arranging the well plates within the insulated receptacle.

22. The method of claim 14, wherein the cell cultures are liver spheroids.

23. The method of claim 14, wherein the insulated receptacle is an insulated box, and the temperature maintenance element comprises a plurality of gel packs heated to a pre-shipping temperature prior to being arranged within the insulated receptacle.

24-40. (canceled)
